(19) United States
(12) Patent Application Publication
(10) Pub. No.: US 2024/0337583 A1
(43) Pub. Date: Oct. 10, 2024

(54) DIFFERENTIATION OF AN INTACT CANCER CELL MEMBRANE WITH RAMAN IMAGING

(71) Applicant: UNIVERSITY OF SOUTH FLORIDA, Tampa, FL (US)

(72) Inventors: Dmitri V. Voronine, Tampa, FL (US); Sharad Ambardar, Tampa, FL (US); Grace Binder, Tampa, FL (US); Phat Huynh, Tampa, FL (US); Dang Nguyen, Tampa, FL (US)

(21) Appl. No.: 18/627,805

(22) Filed: Apr. 5, 2024

Related U.S. Application Data
(60) Provisional application No. 63/494,578, filed on Apr. 6, 2023.

(51) Int. Cl. G01N 15/14; G01N 15/10; G01N 15/1429; G01N 33/483; G06T 5/50; G06T 7/00

(52) U.S. Cl. CPC G01N 15/1468; G01N 15/1429; G01N 33/4833; G06T 5/50; G06T 7/0014; G01N 2015/1006; G06T 2207/10056; G06T 2207/20224; G06T 2207/30024; G06T 2207/30096; G06T 2207/30204

(57) ABSTRACT

Methodology of Surface-Enhanced Raman Spectroscopy with the use of a rough Al substrate to image the biochemical composition of only the plasma membrane (and substantially without the contribution, to the SERS, of the contents of the inside of an imaged cell, thereby differentiating one from another) of intact cancerous cells and intact normal non-cancerous cells. The judiciously generated roughness of the Al substrate resulted in formation of many hot spots that provided a uniform membrane signal. The peak ratios of the resulting Raman signals specific to the cell-membrane only exhibited heterogeneous properties of the membrane with different lipid and protein concentrations at different spatial locations and are identified and can be used as novel cancer biomarkers. The proposed methodology may be extended to studying the nanoscale composition of lipid rafts and protein aggregates in intact biological cells.

### CROSS-REFERENCE TO RELATED APPLICATIONS

This US Patent Application claims the benefit of and priority from the U.S. Provisional Patent Application No. 63/494,578 filed on Apr. 6, 2023, the entire contents of which are hereby incorporated by reference.

### TECHNICAL FIELD

The present invention relates to methodologies of characterizing cancerous cells in a cell culture and, more particularly, to a methodology of distinguishing and discerning an image of only a membrane of a cancerous cell with the use of Raman spectra acquired from the culture cell disposed on a plasmonic and on a passive substrates, and ascertaining the development of cancer is such a cell based on biological markers derived from this image.|

### RELATED ART

Ovarian cancer is one of the most prevalent cancers among women. As a result of carcinogenesis, remodeling of the extracellular matrix as well as changes in the morphology and biochemical composition of the plasma membrane occur. A clear understanding of the plasma membrane relies on developing new techniques for cell surface imaging.

Biomarkers used for oncology often include lipids (such as phosphatidyl serine and cholesterol, among others), which vary with the physiological function of the cell or tissue. While the lipid composition of cancer cells differs from the non-malignant cells of the same type, it is also affected by the type of malignancy. For instance, prior to metastasis, cancer cells have been observed to reduce the cholesterol content to maximize the membrane fluidity due to the central role this steroid plays in maintaining the structural rigidity of the plasma membrane.

Recent studies have demonstrated the activation of protein kinase C (PKC) by phosphatidylglycerol and its involvement in viral envelope formation and transcription and its subsequent role in viral-associated cervical cancer. The molecular models of ovarian cancer are based on the overexpression of certain proteins such as the PKC family. (See, for example, Griner, E. M. et al., in Nat Rev Cancer 2007, 7 (4), 281-294, available at doi.org/10.1038/nrc2110. PKC binds to the inner side of the plasma membrane and forms complexes with other biomolecules, being a target for drugs (see Smalley, T. et al., Small Molecule Inhibitor ζ-Stat, and Its Effects on Invasion Through Decreases in PKC-ζ Protein Expression; in Frontiers in Oncology 2020, 10)

Far-field confocal Raman microscopy has been extensively used for cancer cell imaging. Methods of related art are rooted on imaging a microscale-size laser illumination cell culture volume that necessarily contains information that is spatially averaged over the illuminated portion of the inside of the cell and the illuminated portion of the membrane. In other words, the Raman scattering based image the related art relies on inevitably contains optical data representing both the inside contents and the membrane of a target cell. Conventionally, therefore, it has been assumed that the contribution of the cell membrane to the Raman scattering based image is either negligible (in which case the assessment of characteristics of the cancerous cell has been performed based predominantly on the signal from the contents inside the membrane; and/or the derived results could not be considered reliable at least logically as the contribution of the cell membrane to the image would constitute the unknown amount of optical noise). Notably, as the skilled person will readily understand, this inability of the related art to assess the optical and/or chemical and/or physiological parameters of the cell membrane by itself deprive the methodologies of the related art of the information carried by specific lipids and/or proteins that have affinity to the membrane of the cancerous cell and that can provide information about the development of cancer.

While there appears to be nothing specific that would, in theory, prevent the use of surface-enhanced Raman spectroscopy (SERS) for imaging the cell membrane separately even in intact cells via enhanced near-field excitation, the practical implementation of such method however has been facing a serious challenge of identifying a mechanism with contrast sufficient to distinguish a contribution of the enhanced signal emitted by the membrane from the non-enhanced majority of the cell interior.

Such challenge is recognized in related art as being practically limiting. On a couple of occasions, for example, the use of a single plasmonic nanoparticle (NP) at the tip of a scanning probe microscope—such as AFM in a tip-enhanced Raman scattering (TERS) configuration—was employed; see Hayazawa, N. et al. in Optics Communications 2000, 183 (1), 333-336; available at doi.org/10.1016/S0030-4018(00)00894-4; see also Anderson, M. S., in Applied Physics Letters 2000, 76 (21), 3130-3132; available at/doi.org/10.1063/1.126546). Proof-of-concept experiments of TERS membrane imaging in cells immediately confirmed challenges in repeatability, tip stability and limited signal enhancement from a single plasmonic particle (see, for example, Böhme, R. et al., Journal of Raman Spectroscopy 2009, 40 (10), 1452-1457, doi.org/10.1002/jrs.2433). The advantage of SERS substrates for membrane imaging is the increased intensity and number of hot spots in the excitation volume to address these challenges. For example, previous work used plasmonic substrates made of randomly aggregated colloidal Ag NPs to obtain SERS spectra of the membrane in live red blood cells. Zito, G. et al. (in Nanoscale 2015, 7 (18), 8593-8606; doi.org/10.1039/C5NR01341K) used, for example the resonance Raman signal of heme as a contrast mechanism and demonstrated a large density of plasmonic hot spots (˜10⁴ μm⁻²) resulting in a spatially flat enhancement factor (EF) and showed large EF values approaching single-molecule sensitivity and high-quality imaging.

Plasmonic material such as Au or Ag, for example, which are characterized by the highest values of the figure-of-merit defined by a ratio of the real part of the frequency-dependent dielectric function to the imaginary part of such function, F_{LSR}=−ε₁/ε₂, are conventionally selected for plasmonic application over the near-infrared and/or visible spectral range. according to their efficiency in the generation of high-quality propagating surface plasmon polaritons. While sp-metals such as aluminum, Al, has been proposed as a candidate for US surface, their affinity to aggressive oxidation and reactivity have been described in related art as practically limiting their use in plasmonic applications (see Sanz, J. M. et al., J. Phys. Chem. C 117, 19606, 2013; Gutierrez, Y. et al., J. Appl. Phys. 128, 080901, 2020), let alone biomedical application.

### SUMMARY OF THE INVENTION

Embodiments of the invention provide an imaging method that includes at least the steps of acquiring a first optical image (formed by Raman scattering of excitation light at a first portion of a biological cell culture carried by an aluminum substrate), acquiring a second optical image (formed by Raman scattering of the excitation light at a second portion of the biological cell culture carried by a passive substrate), and—based on judicial transforming these first and second optical images with the use of forces images of the first and second portions of the cell culture—setting apart or differentiating between a first Raman scattering signal contributed to the observe Raman scattering by an interior of a cell of the biological cell culture and a second Raman scattering signal contributed to the observed Raman scattering only by a membrane of the cell. (Here, the used force images are those acquired with the use of a force microscope apparatus.) In at least one specific implementation of the method, the biological cell culture may be a culture of cancerous cells while in a related implementation two biological cultures are used—one of cancerous cells and another of normal, non-cancerous cells. Alternatively or in addition—and substantially in every implementation—the method may be carried out without impairing the membrane and/or without separating the membrane from an interior of a cell being imaged. The process of transforming given two optical images generally includes subtracting one of the two optical images from the other and/or normalizing at least one of the two optical images by a height of a peak of a corresponding force image of the force images acquired with the force microscope apparatus. Alternatively or in addition, and substantially in every implementation, the method may include a step of generating a target image (that represents only a membrane of a cell of the biological culture) based on the results of the process of transforming. When such step of generating a target image is present, the target image is optionally that of plasma membrane of an intact cancer cell and/or—when the biological cell culture is that of cancerous cells, the step of generating includes generating a cancerous target image that represents only an intact membrane of a cancerous cell. Optionally—and at least in one implementation of the method—when the biological cell culture is a cell culture of cancerous cells, the step of generating includes generating a cancerous target image representing only a membrane of a cancerous cell while the method additionally includes the following steps: (a) acquiring a third optical image (formed by Raman scattering of excitation light at a third portion of an auxiliary biological cell culture carried by the aluminum substrate, the auxiliary biological culture being a culture of non-cancerous cells), (b) acquiring a fourth optical image (formed by said Raman scattering at a fourth portion of the auxiliary biological cell culture carried by a passive substrate), (c) differentiating between a third Raman scattering signal contributed to the overall observed Raman scattering by an interior of an auxiliary cell of the auxiliary biological cell culture and a fourth Raman scattering signal contributed to the overall Raman scattering by a membrane of the auxiliary cell based on transforming the third and fourth optical images with the use of force images (produced with the use of a force microscope) of the third portion and the fourth portion, and (d) generating a non-cancerous target image representing only a membrane of the auxiliary cell of the auxiliary biological cell culture. (At least in the latter case, the method may yet additionally include a step of generating indicia of protein activity in the cancerous cell based at least on comparison of respectively corresponding lipid-to-protein ratios that are derived from the cancerous target image and the non-cancerous target image.) Alternatively or in addition—and substantially in every implementation of the method, the target image is an image representing only a membrane of an intact cell of a given biological culture, while the method additionally includes at least the following steps: (i) assigning a first lipid-to-protein ratio, identified based on the target image in a first portion of the membrane, as a first biomarker, (ii) assigning a second lipid-to-protein ratio, identified based on the target image in a second portion of the membrane that is different from the first portion of the membrane, as a second biomarker, and (iii) based at least on the use of such first and second biomarkers, identifying changes in a biomolecular composition of the membrane that are specific to an identified cancer; and optionally—generating indicia of the identified cancer based at least on results of said identifying. Furthermore, in at least one specific embodiment the method may include a step of generating the aluminum substrate by aggregating aluminum nanoparticles with random distribution of dimensions of up to and/or in excess of about 50 nm on a supporting surface to increase enhancement factor of Raman scattering at the first portion of the biological cell culture.

### BRIEF DESCRIPTION OF THE DRAWINGS

The invention will be more fully understood by referring to the following Detailed Description of Specific Embodiments in conjunction with the Drawings, of which:

FIGS. 1A, 1B, 1C: Al SERC imaging of an intact cell membrane. FIG. 1A: a schematic diagram of the Al SERS imaging of an intact cell membrane. Far-field illumination at 532 nm was used to excite both cell interior and membrane on Al and Si substrates, while near-field of Al NPs enhanced the membrane signal on the Al substrate only; FIG. 1B: Optical images of SHT290 cells on the Al substrate; FIG. 1C: optical image of the SHT290 cell on the Si substrate;

FIGS. 2A, 2B, 2C: Morphological characterization of a rough Al substrate. FIG. 2A: AFM height image; FIG. 2B: Height profile across the solid line 210 in FIG. 2A; FIG. 2C: Zoomed-in height profile enclosed in black dashed area of FIG. 2B. Ellipses in FIG. 2C outline the positions of Al NPs;

FIGS. 3A, 3B, 3C, 3D, 3E, 3F, 3G, 3H, 3I, and 3G: Characterization of SHT290 normal cells. FIG. 3A: on a Si substrate; FIG. 3B: on an Al substrate; Correlated AFM height and Raman intensity profiles of SHT290 cells on the SI substrate (FIG. 3E) and Al substrate (FIG. 3F) that correspond to the arrows in AFM height images (FIGS. 3C and 3D, respectively). White dashed lines in FIGS. 3C, 3D represent the cell outlines. Raman spectra on the nucleus (FIG. 3I) and cytoplasm (FIG. 3J) areas that correspond to the highlighted areas N and C in Raman maps on Si (FIG. 3G) and Al (FIG. 3H) substrates;

FIGS. 4A, 4B, 4C, 4D, 4E, and 4F: Raman spectra (blue lines) and peak fittings (green lines) with a linear combination of four Lorentzian functions (red lines) in fingerprint and high wavenumber regions for ES-2 cancer cells nucleus and cytoplasm-proximal areas on Al (FIGS. 4A, 4B) and Si (FIGS. 4C, 4D) substrates and their difference (labelled “Al—Si”; FIGS. 4E, 4F). The peaks are numbered according to the assignment in Table 1.

FIG. 5 includes multiple graph-windows (a), (b), (c), (d), (e), (f), (g), and (h), each presenting a bar diagram. FIG. 5. Peak ratios of the vibrational bands normalized by the reference peaks at 1449 cm⁻¹ (graph-windows (a)-(d), (g), (h)) and at 1328 cm⁻¹ (graph-windows (e) and (f)).

FIGS. 6A, 6B, 6C, 6D, and 6E: characterization of cells. Kelvin probe force microscopy (KPFM) imaging of ES-2 cells on Si (FIG. 6A) and Al (FIG. 6B) substrates, and of SHT290 cells on Si (FIG. 6C) and Al (FIG. 6D) substrates. FIG. 6E: Surface potential fluctuations of SHT290 (N1-N3) and ES-2 (C1-C3) cells calibrated using an Au substrate.

FIGS. 7A, 7B, 7C, 8D, 7E, and 7F: Sketches of cells on Si (FIG. 7A) and Al (FIG. 7D) substrates. AFM height profiles of SHT290 cells on Si (FIG. 7C) and Al (FIG. 7F) correspond to the arrows in AFM height images (FIG. 7B) and (FIG. 7E), respectively. White dashed lines in (FIG. 7B) and (FIG. 7E) represent outlines of the cells.

FIGS. 8A, 8B, 8C, 8D, 8E, 8F, 8G, 8H, 8I, 8J: Optical images of ES-2 cancer cells on Si (FIG. 8A) and Al (FIG. 8B) substrates. Correlated AFM height and Raman intensity profiles of ES-2 cells on Si (FIG. 8E) and Al (FIG. 8F) that correspond to the arrows in AFM height images (FIG. 8C) and (FIG. 8D), respectively. White dashed lines in (FIG. 8C) and (FIG. 8D) represent the cell outlines. Raman spectra on nucleus (FIG. 8I) and cytoplasm (FIG. 8J) areas that correspond to the highlighted N and C areas in Raman maps on Si (FIG. 8G) and Al (FIG. 8H) substrates.

FIGS. 9A, 9B, 9C, 9D, 9E, and 9F: Raman spectra (blue lines) and peak fittings (green lines) with a linear combination of four Lorentzian functions (red lines) in fingerprint and high wavenumber regions for SHT290 normal cells nucleus and cytoplasm-proximal areas on Al (FIGS. 9A, 9B) and Si (FIGS. 9C, 9D) substrates and their difference (labelled “Al—Si”; FIGS. 9E, 9F). The peaks are numbered according to the assignment in Table 1.

FIGS. 10A, 10B: Raman spectra of ES-2 cells from nucleus areas on Au (FIG. 10A) and glass (FIG. 10B) substrates.

FIGS. 11A, 11B, 11C, 11D: Average peak ratios of the normalized vibrational bands from the areas shown in FIGS. 3A-3G. Two additional areas of SHT290 cell on Si (FIG. 11E) and Al (FIG. 11F) substrates: nucleus-proximal (A1, A2) and cytoplasm-proximal (B1, B2) areas are shown in AFM height images of FIGS. 11E, 11F.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another.

### DETAILED DESCRIPTION

### General

Raman spectroscopy is a substantially label-free non-invasive method of chemical analysis, but it has been limited by weak signals from the membrane as compared to signals representing the bulk interior of the cells. Certain molecules have such prominent vibrational characteristics that they can be considered and/or used to be cancer biomarkers. Considering that the plasma membrane of a cancer cell displays differences in lipid and protein composition which distinguish them from a healthy cell, the inability to derive the Raman-scattering-based information representing the cell membrane only and/or overlooking such information deprives the user from the ability to characterize such molecules that have affinity to the cell membrane and, as a result, acquire diverse information about the development of cancer in the cells.

Based on the discussion below, the skilled person will appreciate that the turning point of the discussed developments include generation of a Raman scattering based image representing only a membrane portion of the biological cell—and, in particular, a cancerous biological cell.

In that, persisting problems of the related art that manifest in substantial inability of the existing methods to assess the characteristics of the cancerous cell materials (such as lipids and/or proteins) that have specific affinity to and for that reason attaching to the cell membrane) with the use of Raman spectroscopy—and, for that reason, in missing the cancer-related information represented by such cell materials are solved by embodiments of the present invention. The implementations of the idea of the invention embody a methodology is disclosed for the novel use of Al-surface enhanced Raman Spectroscopy for pointedly devising an image substantially exclusively representing plasma membranes of intact cancer cells (that is under conditions when the physical separation of the cell membrane from the contents of the cell and, therefore, the destruction of the cell, is avoided) with the assistance of the cell-carrying substrate made of aluminum, in stark contradistinction with approaches used in related art. Furthermore, the surface of the employed Al substrate was intentionally roughened by forming such substrate via deposition of Al nanoparticle (NPs) that were randomly sized, to ensure broad range of morphologies to increase the strength of plasmonic resonance that such substrate could provide to the SERS-based imaging in a situation when a substantially flat (conventionally—with less than about 0.5 nm roughness) plasmonic substrates are used. The AFM height measurements were employed as a contrast mechanism to separate the Raman signals of the membrane from those received from the cell interior. In the process of such characterization, the chemical composition of some chosen target cells (ES-2 clear cell ovarian carcinoma cells and immortalized normal endometrial stromal cell line SHT290) were compared.

The entire contents of the publication by Ambardar, S. et al., (titled “Surface-enhanced Raman imaging of intact cancer cell membrane on a rough aluminum substrate”, in the J. of Raman Spectroscopy, 2023; 54:940-949), as well as other publications identified in this disclosure, is incorporated herein by reference.

### Materials, Methods, Experimental Setup

Aluminum substrates were prepared by depositing a thin layer of Al NPs on standard glass slides using a plasma sputtering system. (The diameter of the Al target was 50 mm and the thickness was 3 mm; the 1.5-2 mTorr pressure, 8.5 sccm Ar flow, 100 mA current and 450 V voltage were use during the sputtering process.)

Cell cultures were grown on different substrates—on the deposited Al substrate and on a Si substrate—under substantially identical conditions. The human ovarian cancer ES-2 CCOC cell line (American Type Culture Collection, USA) was cultured in MCDB 131: Media 199 (1:1 ratio) and McCoy's medium, respectively, supplemented with 10% FBS, 100 units/ml of penicillin and 100 μg/ml of streptomycin. The immortalized normal human endometrial stromal cell line, SHT290 (Kerafast, USA) was maintained in F12K: Media 199 (1:1 ratio) and supplemented with 5% FBS, 0.1% Mito, 2 μg/ml of human insulin, 100 units/ml of penicillin and 100 μg/ml of streptomycin. Each cell culture was passaged less fewer than 10 times. Cell cultures were maintained in an incubator at 37° C. and 5% CO₂ atmosphere. Cells were cultured on aluminum substrates as described above, on Si substrates with 300 nm SiO₂ coating, on glass and on atomically flat Au substrates (Tedpella). Cells were washed with phosphate-buffered saline (PBS) before and after fixation to remove residual media or fixative solution, respectively. Samples were fixed using 4% paraformaldehyde (PFA) for 15 min at room temperature. After washing, fixed cells were stored in PBS with 0.01% sodium azide. Samples were rinsed with methanol prior to analysis.

Optical, Raman, atomic force microscopy (AFM), and Kelvin probe force microscopy (KPFM) measurements were performed using our setup based on the tapping mode AFM (as discussed in, for example, Ambardar, S. et al. Nanoscale 2022, 14 (22), 8050-8059; doi.org/ch, the disclosure of which is incorporated herein by reference). In particular, AFM and Raman imaging experiments were performed using a confocal microscope (LabRam, Horiba Scientific) coupled to a scanning probe microscope (OmegaScopeR, Horiba Scientific) with 532 nm laser excitation focused using an objective lens (NA=0.9). AFM measurements were performed in tapping mode with about 20 nm average tip-sample distance. The samples were checked for thermal damage after every experiment by repeated AFM imaging to confirm that no significant changes in AFM sample morphology were occurring. KPFM measurements were performed using a conductive Au-coated AFM tip. The radius of the laser focal spot was about 500 nm. Using a backscattering configuration with a 532 nm edge filter, the scattered signals were collected and detected by a spectrometer with 600 g/mm grating coupled to a CCD camera. Raman maps were obtained using an acquisition time of 5 s and 0.5 mW laser power. Background subtraction in Raman spectral processing was done using a 7^{th} order polynomial fitting. All experiments were performed under ambient conditions at room temperature.

### Experimental Results

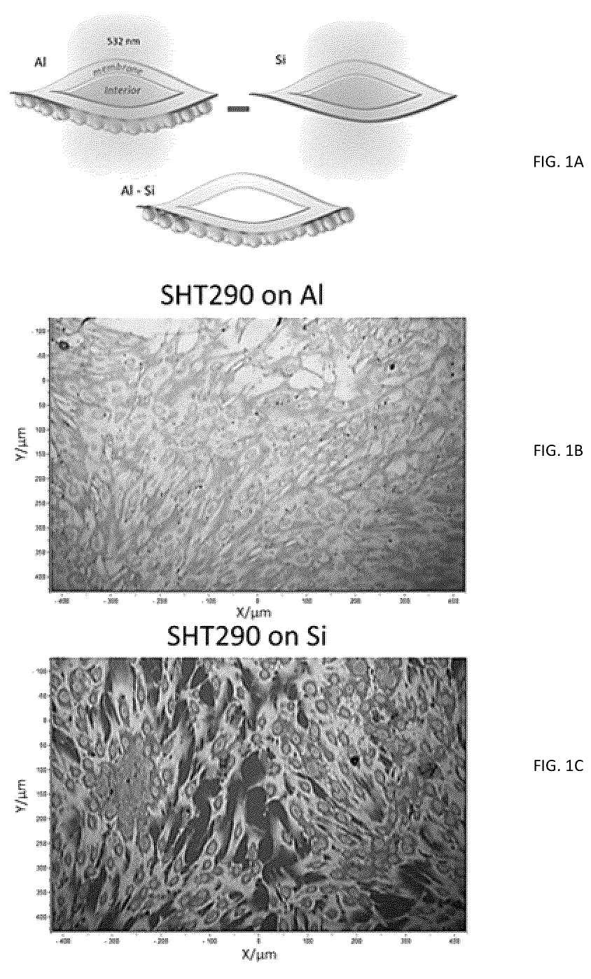

The schematic diagram of the membrane imaging approach is shown in FIG. 1A. Under the 532 nm laser excitation, confocal Raman images were obtained for the substantially identical cell cultures—one on the Al substrate, another on the Si substrate—each of which contained both a signal formed at a cell membrane and that at a cell interior portion. As a skilled person will understand, due to the SERS effect of the Al substrate, a cell membrane of a given cell adjacent the Al substrate had an additional Raman signal enhancement due to the proximity to the Al nanoparticle (NP) surface. The plasmonically enhanced signal from the membrane only was judiciously procured by finding a difference between these two images (that is, a difference between the image of a first portion of the cell culture disposed on the AL substrate and that of a second portion of the cell culture disposed on the Si substrate), see FIG. 1A

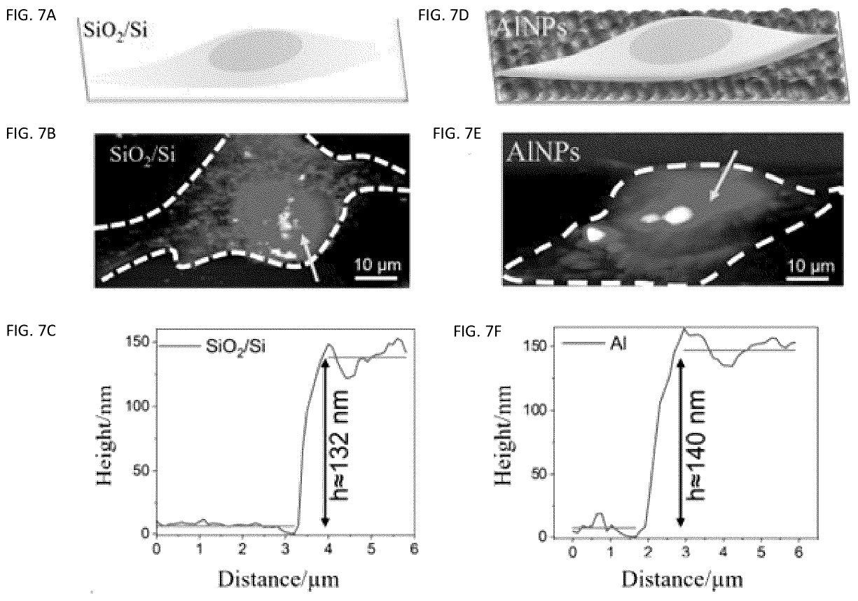

To properly spatially calibrate the procedure of image transformation and to differentiate a portion of the SERS signal representing substantially only a membrane of the cell, the subtraction of the two Raman signals was performed by selecting substantially the same height areas using the AFM height profiles of both cancer and normal cell types as shown in FIGS. 7A, 7B, 7C, 7D, 7E, and 7F. For example, the areas of the nucleus-proximal part of the SHT290 cells on Si and Al substrates had an average height of 136 nm that was determined from the profiles in FIGS. 7C and 7F. The same far-field excitation volume was assumed that was determined from the AFM height profiles corresponding to the same amount of material in the interior of cells. Therefore, the subtraction of the Raman signals normalized by the AFM height resulted in the separated Raman signal of the cell membrane on Al substrate. These measurements were performed on normal SHT290 and cancer ES-2 cells.

It is appreciated, therefore, that a practical implementation of the methodology of the invention included acquisition of a first optical image (with the use of Raman scattering of excitation light at a first portion of a biological cell culture carried by an aluminum substrate) and acquisition of a second optical image (with the use of Raman scattering of the excitation light at a second portion of the biological cell culture carried by a passive substrate—that is, the substrate substantially devoid of plasmonic properties, such as—in a non-limiting example—a silica-on-silicon substrate) to ascertain that contribution to the overall Raman scattering signal that is brought about only by a membrane of the cell being imaged. Such assessment is based on transforming the first and second optical images with the use of force images (for example, images acquired with the use of a fore microscope) of the first portion and the second portion of the cell culture. As a result, the assessment of the “membrane-only” image was performed without impairing the membrane of a cell being imaged and/or without separating the membrane from an interior of the cell, in stark contradistinction with related art. As discussed below, when the imaged cell culture included cancerous cells, such assessment enabled the user to define specific novel biomarkers via identifying ratios representing two groups of molecules (proteins, lipid) that have affinity to and that are attached from the inside or outside of the intact cell to the membrane and—in comparison with the corresponding ratios produced by these two groups of molecules at the membranes of a non-cancerous cell—to make conclusions about the developments of cancer as represented only by these two groups of molecules. In doing so, as discussed below, different regions of (cancerous and/or non-cancerous) cells were investigated: the one proximal to the nucleus and the one proximal to the cytoplasm.

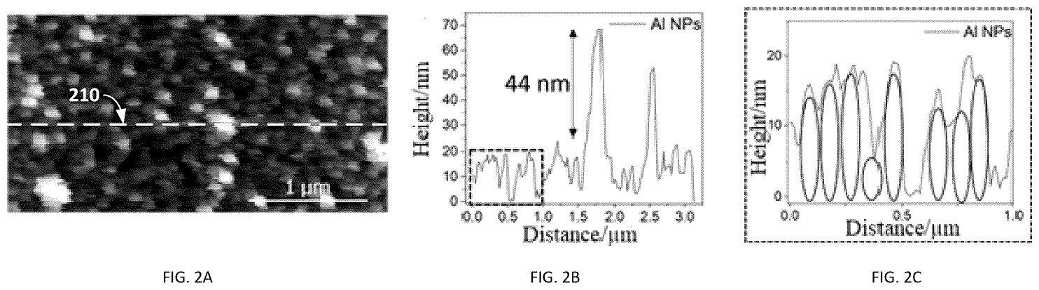

AFM imaging was additionally used to investigate the roughness of the Al substrate formed by depositing AL NPs having randomly varying dimensions on a supporting surface. FIG. 2A provides the AFM height image revealing the presence of Al NPs with the average height of ˜50 nm as determined from the AFM height profiles (FIGS. 2B, 2C). The enhancement factor (EF) provided by such substrate for the Raman scattering was then estimated by considering the near-fields of hot spots. Electromagnetic hot spots are localized areas in the gaps between plasmonic nanoparticles that generate the largest EFs. Due to the exponential near-field decay, the hot spot size is typically less than about 10 nm. The conventional definition of EF was used (see, for example, Ru, E. L. at al., in Principles of Surface-Enhanced Raman Spectroscopy: And Related Plasmonic Effects; Elsevier, 2008):

$$EF = ( \frac{{I}_{Al}}{{I}_{Si}} - 1 ) \times \frac{{V}_{FF}}{{V}_{NF}} , \qquad ( 1 )$$

where I_{Al} and I_{Si} are the integrated intensities of Raman signals received from the cells on the Al and Si substrates, respectively, and where V_{FF} and V_{NF} correspond to the far-field (FF) and near-field (NF) excitation volumes (the ratio of which gives the normalization factor that accounts for a different number of molecules generating the Raman signals). Assuming disk-shaped excitation spots with FF and NF disk heights of h_{FF}=150 nm (SHT290 cell height, see below) and h_{NF}=10 nm (near field decay length), and the same excitation spot radii for FF and NF, R_{FF}=R_{NF}=1 μm, the EF of approximately 30 was assessed.

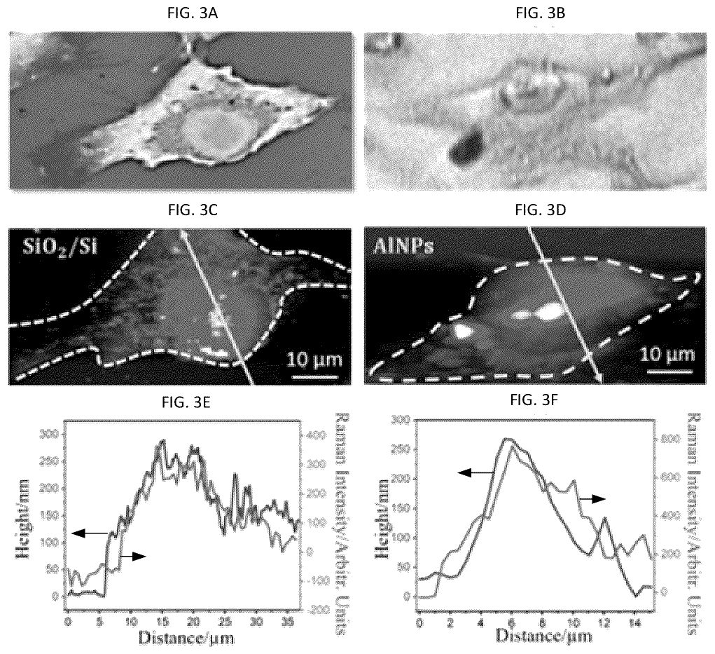

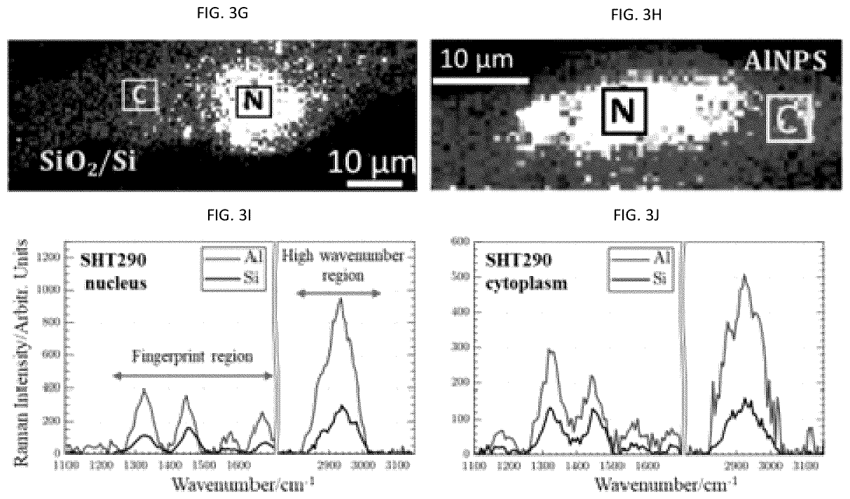

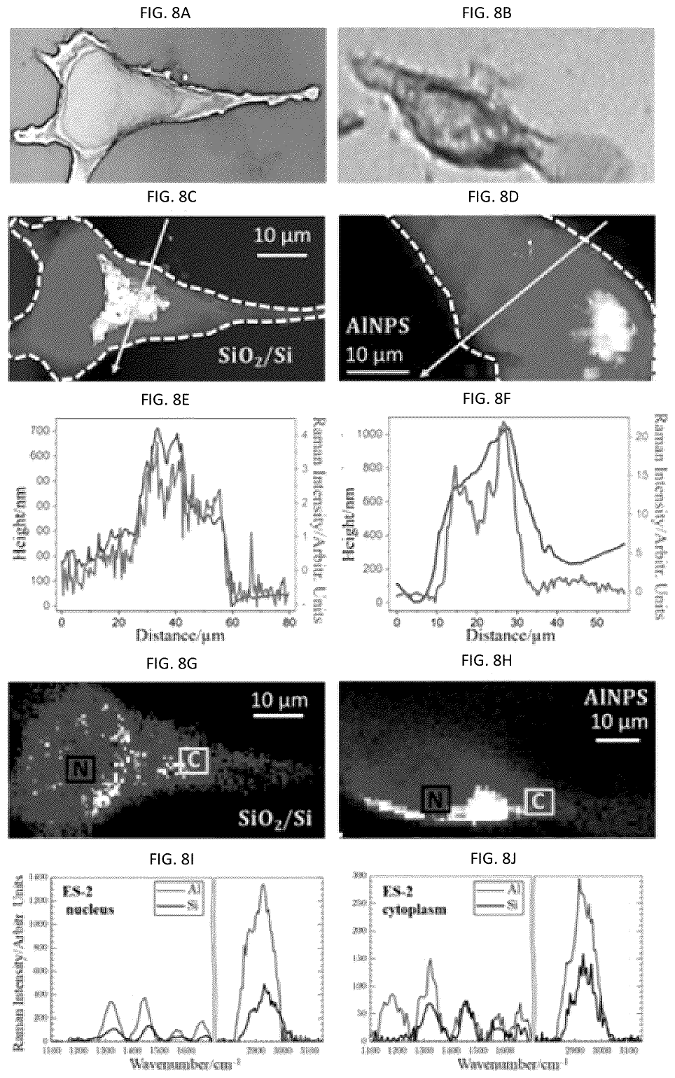

Optical (FIGS. 3A, 3B), morphological (FIGS. 3C, 3D) and spectroscopic (FIGS. 3E, 3F, 3G, 3H, 3I, 3J) analysis of SHT290 normal cells on Si and Al substrates was carried out. The corresponding ES-2 cancer cell data are shown in supplementary FIGS. 8A through 8J. The AFM and optical images were used to identify the location of the nucleus inside the cells. The Raman maps in FIGS. 3G, 3H were obtained by integrating the Raman intensities in the high frequency range shown in FIGS. 3I, 3J.

As indicated in FIGS. 3G, 3H, rectangular areas C and N in images were identified that corresponded to the cytoplasm and nucleus areas, respectively. Since the Raman signal linearly depends on the number of molecules, areas of similar height for the quantitative comparison of the Raman signals on different substrates were chosen. The AFM height and Raman intensity profiles of SHT290 cells on the Si substrate (FIG. 3E) and on the Al substrate (FIG. 3F) displayed strong correlation, thereby confirming the linear dependence. The Raman spectra of the nucleus (FIG. 3I) and cytoplasm (FIG. 3J) proximal areas that corresponded to the highlighted areas N and C in Raman maps on Si (FIG. 3G) and Al (FIG. 3H) substrates exhibited clear increase in a signal originated from the cells placed on the Al substrate due to the surface enhancement of the signal generated at the cell membrane. Here, two spectral ranges were considered, namely, the fingerprint region of 1100-1700 cm⁻¹ and the high wavenumber region of 2700-3150 cm⁻¹. The high wavenumber region includes four main bands from lipids and proteins, while the fingerprint region is more complex and contains additional contributions from other biomolecules such as RNA and DNA. A detailed analysis of these spectra is presented below.

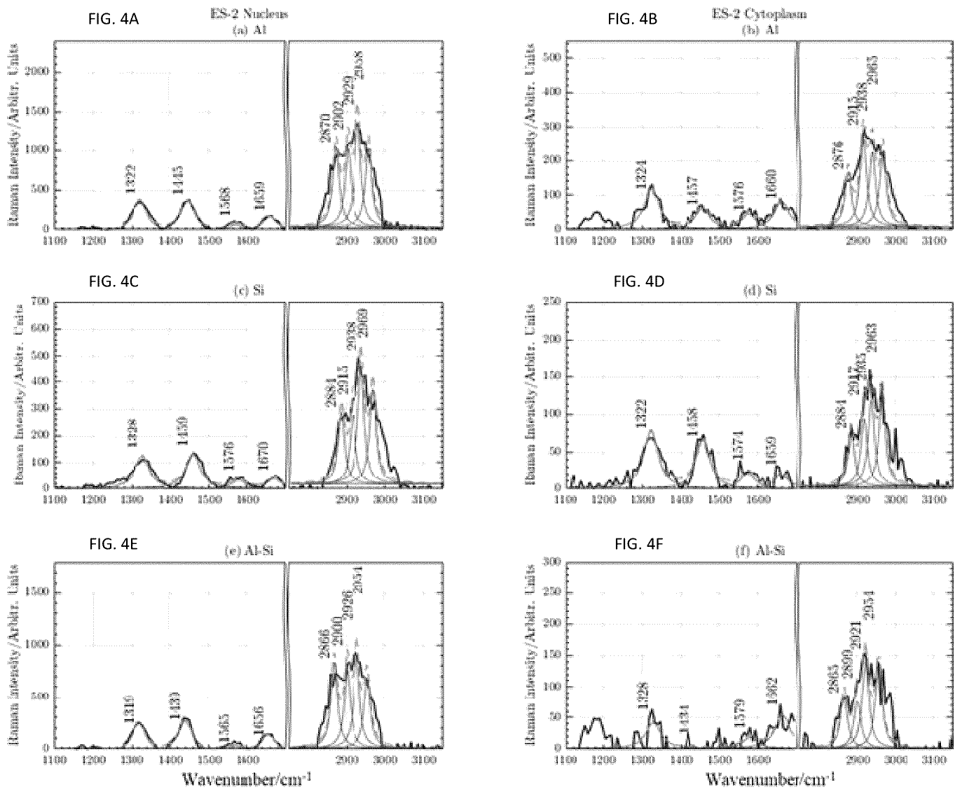

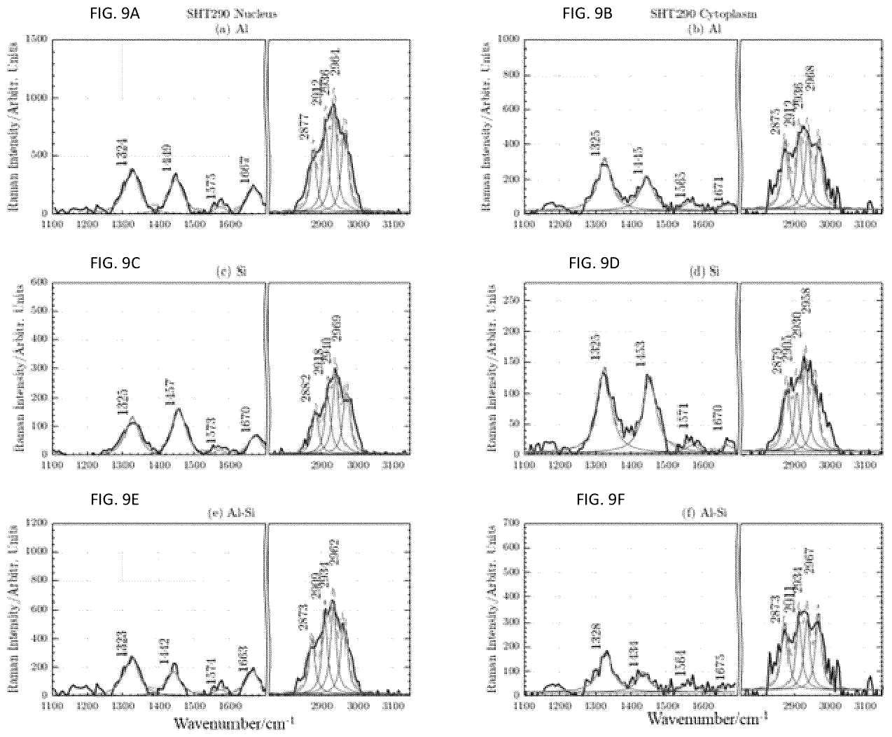

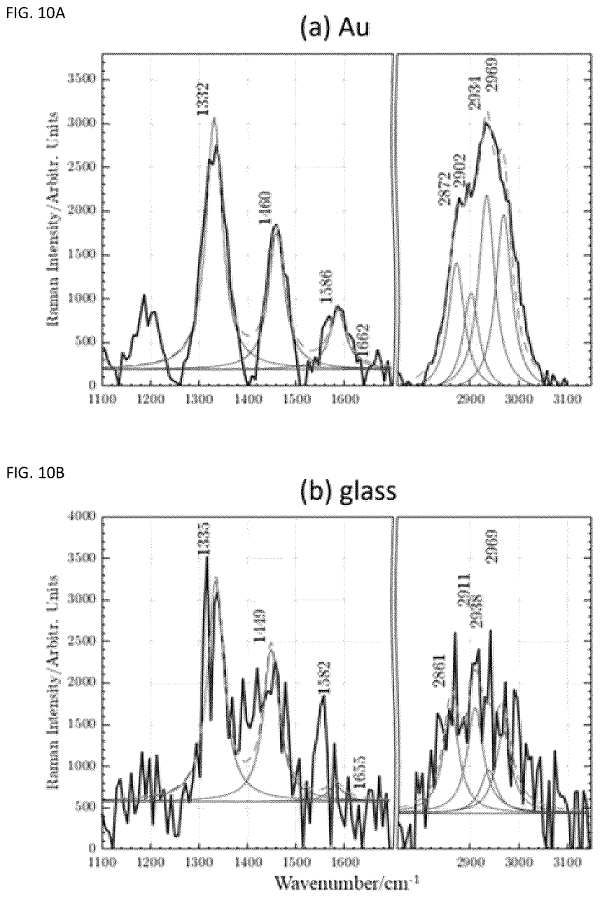

Peak fitting of the Raman spectra was further performed using the MATLAB peak-fitting toolbox for spectroscopic data analysis with a linear combination of Lorentzian functions. Here, a simplified four-peak model was applied to each of the two regions as shown in FIGS. 4A, 4B, 4C, 4D, 4E, and 4F. The Raman shift range of ±15 cm⁻¹ was used for each peak assignment in Table 1. FIGS. 4A-4F show the Raman spectra (blue lines) and peak fittings (green lines) for ES-2 cancer cells nucleus and cytoplasm-proximal areas on Al (FIGS. 4A, 4B) and Si (FIGS. 4C, 4D) substrates and their difference (labeled “Al—Si”; FIGS. 4E, 4F). The latter corresponds to the SERS signal from the cell membrane, as the skilled artisan will readily appreciate. The corresponding Raman spectra and fittings for SHT290 normal cells are shown in FIGS. 9A, 9B, 9C, 9D, 9E, 9F.

Table 1 shows the peak assignments for the Raman spectra of ES-2 cells on a Si substrate based on the previously reported data on Raman spectra of biological cells and tissues (Movasaghi, Z., et al., in Applied Spectroscopy Reviews 2007, 42 (5), 493-541). Similar peaks were observed in ES-2 and SHT290 cells on all substrates with Raman shift changes within the range of ±15 cm⁻¹. Therefore, Table 1 was used to describe all samples in this work.

TABLE 1
Peak assignments of major molecular components of ES-2
cells on Si substrate: P—protein; L—lipid.

| | |
|---|---|
| Wavenumber/cm⁻¹ | Assignment |

Fingerprint region

| | |
|---|---|
| 1325 | Amide III, C—H deformation (P) |
| 1449 | CH₂, CH₃ deformation (L/P) |
| 1572 | Amide II, C═C stretch (P) |
| 1660 | Amide I, C═O stretch (P) |

High wavenumber region

| | |
|---|---|
| 2876 | CH₂ symmetric stretch (L) |
| 2911 | CH₂ asymmetric stretch (L) |
| 2925 | CH₃ symmetric stretch (L/P) |
| 2954 | CH₃ asymmetric stretch (L/P) |

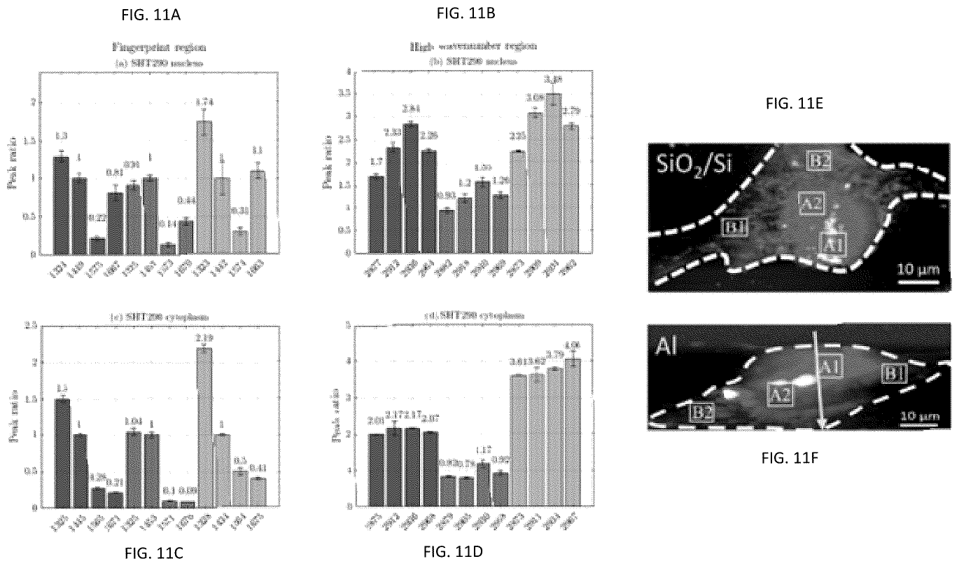

The ratios of the relative concentrations of biomolecules were obtained by analyzing the peak ratios of the vibrational bands in FIG. 5 (graph-windows (a) through (h)). The data normalization was performed on the baseline-subtracted Raman spectra using the reference peak at 1449 cm⁻¹ because this peak was well isolated and was present with high intensity in all data except for the substrate-adjacent ES-2 membrane of the cytoplasm-proximal area (FIG. 4A. 4B, 4C, 4D, 4E, 4F). In the latter case, the reference peak at 1328 cm⁻¹ was used for the normalization in graph-windows (a) and (f) of FIG. 5. FIG. 5 as a whole shows different chemical compositions of the plasma membrane of ES-2 and SHT290 cells. For example, the 1449/1660 cm⁻¹ peak ratio is 1.72 and 0.97 for the ES-2 and SHT290 N-proximal areas, respectively. However, this ratio is reversed to approximately 0 and 2.56 for the corresponding C-proximal areas, respectively. The 1449/1660 cm⁻¹ peak ratio is proportional to the relative lipid/protein (LIP) concentration. Therefore, our results indicate the different L/P compositions in different parts of the membrane. The C-proximal area of ES-2 cells has an increased concentration of proteins that are tentatively assigned to the actin cytoskeleton. However, the N-proximal area has more lipids in ES-2 cells relative to proteins. This may be due to the overproduction of actin by cancer cells for increased cell migration. The opposite trends in the normal SHT290 cells indicate lower migration and proliferation activities. These results are further supported by the high wavenumber region 2876/2925 cm⁻¹ peak ratio which is similarly related to the L/P ratio. The peak ratios in FIG. 5 had less than 5% variations compared to similar areas. FIGS. 11A, 11B, 11C, 11D, 11E, and 11F show the average peak ratios and standard deviations of the additional two N- and C-proximal areas on SHT290. Similar peak ratios were observed.

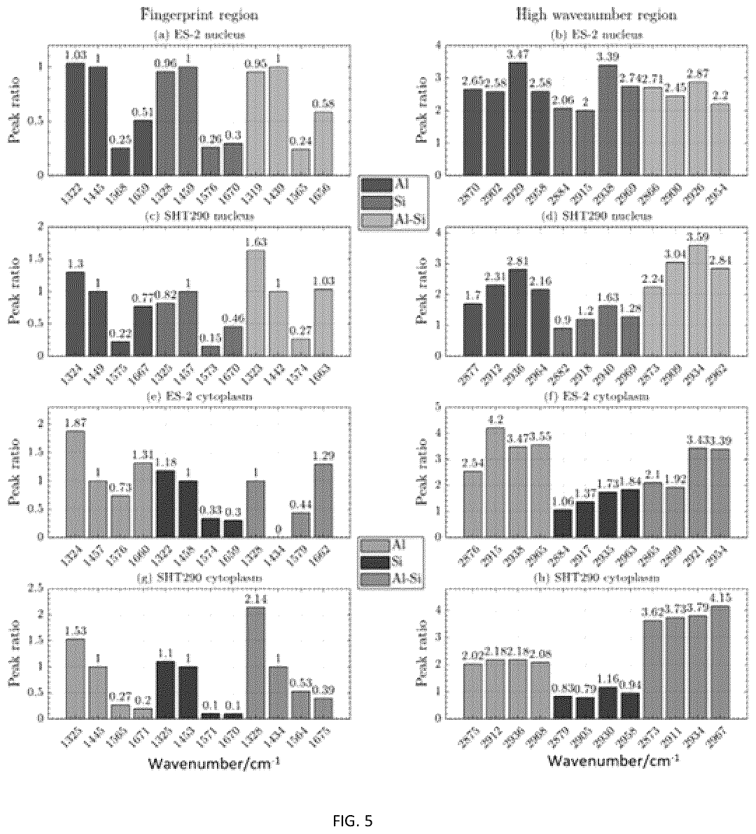

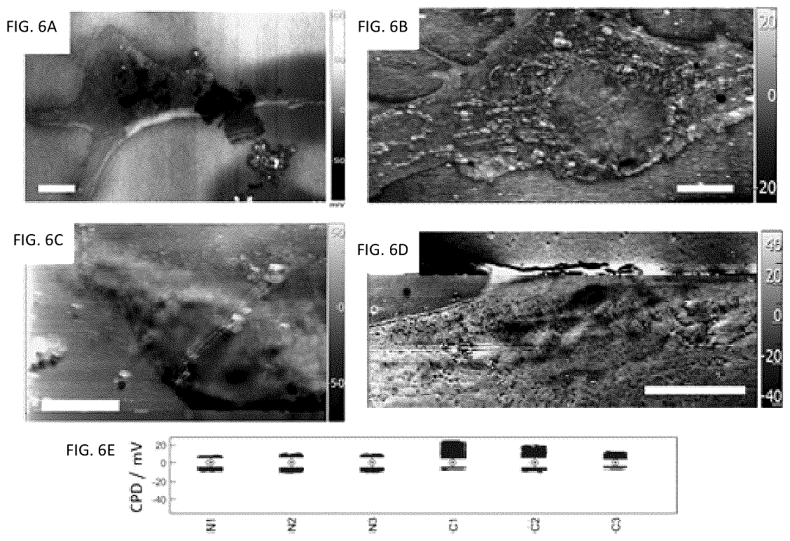

Additionally, the analysis of the cell membrane surface potential was performed by KPFM imaging as described in the Methods section of this disclosure. The plasma membrane resting potential was previously shown in related art to undergo depolarization in cancer cells. Various mechanisms of membrane potential regulation involve cell signaling pathways mediated by the disrupted activities of ion channels, pumps, and transporters. The membrane potential, therefore, can be viewed as an important bioelectric marker that reflects the changes in cellular activities. KPFM is a nanoscale electrostatic force imaging technique based on the contact potential difference (CPD) between a scanning probe tip and sample. FIGS. 6A, 6B, 6C, 6D, and 6E illustrate CPD images of the ES-2 cells on Si (FIG. 6A) and Al (FIG. 6B) substrates, and the SHT290 cells on Si (FIG. 6C) and Al (FIG. 6D) substrates. The nanoscale distribution of the surface potential calibrated on an Au substrate shows spatial fluctuations with a larger range of depolarization in cancer ES-2 cells (C1-C3 in FIG. 6D), which is substantially independent of the substrate. This supports the conclusion that the observed differences between the cancer and normal cells in this work do not originate from different cell metabolism on different substrates.

The related art disclosed that the membrane contribution to the confocal Raman spectra of cancer cells was negligible without using any surface enhancement (see, for example, Shafer-Peltier, K. E. et al. in Journal of Raman Spectroscopy 2002, 33 (7), 552-563). This disclosure presents the very first implementation of Al plasmonics to SERS imaging of cancer cells, resulting in acquisition of uniform Raman images with high correlation of morphological AFM profiles. This indicates a large number density of plasmonic hot spots within a laser focus that is required for membrane imaging. By subtracting the signals acquired with the use of the passive substrate (in this case—Si substrate, used due to its relatively low background as compared to the more commonly used glass for cell cultures) from those acquired with the use of the Al substrate, the judicious identification of and enhanced contribution of the membrane-only signals became possible. (Notably, for the sake of comparison performed measurement on glass substrates showed Raman signals that were too weak for the membrane-only analysis.)

In all experiments, the enhancement of EF of about 15 to 30 was observed on both normal (non-cancerous) cells and cancer cells by comparing the signals obtained with the use of Al and Si substrates over the whole available spectral range. These results evidence that the electromagnetic (EM) SERS mechanism is the primary mechanism for the SERS, where the same number of molecules generated the Raman signals based on AFM height normalization. The differences between the FF Raman and SERS spectra are assigned to the different chemical compositions of the cell membrane and the cell interior. For example, the switching of the 1449/1660 cm¹ peak ratios of lipids and proteins was different in normal and cancer cells (see FIG. 5). This substantially established that the resulting difference spectra are at least in part due to SERS in addition to possible difference in cell metabolism.

Furthermore, as discussed, the comparison of the intensity of Raman spectra of the ES-2 cells on the Al (structured as discussed) and those obtained with the use of the substantially atomically flat Au substrates (with less than ˜0.5 nm roughness). Such non-plasmonic, flat Au substrates substantially did not have any surface plasmon resonances while, at the same time, demonstrating stronger Raman spectra compared to both Si and Al substrates (see FIGS. 10A, 10B). This enhancement was attributed 11to the chemical mechanism (CM) of the flat Au substrate because the plasmonic EM mechanism is absent in the flat Au surface without any nanostructures. The overall results demonstrate that the SERS effect may be observed in both flat and rough metallic surfaces based on the presence of the two SERS mechanisms. Even though the Al substrate may be considered to be less efficient than the flat Au substrate for the purposes of the SERS, such AL substrate has the advantage of being at least less expensive under otherwise equal circumstances.

Notably, while cell morphology methods have commonly been used by pathologists for cancer diagnosis based on the nucleus-to-cytoplasm ratio in related art, this type of analysis considers specifically the bulk properties of the cells. In stark contradistinction with related art, the presented discussion introduced the corresponding surface analysis based on the properties of only the plasma membrane areas that are located in proximity to the nucleus or away from the nucleus, (which may be referred to as the cytoplasm-proximal area). The presented analysis reflects changes in the global relative concentrations of lipids and proteins in the plasma membrane.

While not necessarily expressly shown in the Figures, implementation of at least some of embodiments of the invention may require the use of a processor controlled by instructions stored in a memory—for example, for collection of optical data characterizing the operation of an apparatus of the invention. Such memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

Understandably, a computer program product containing program code(s) embodying and/or governing the operation of at least one implementation of the idea of the invention remain within the scope of the invention.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes. Other specific examples of the meaning of the terms “substantially”, “about”, and/or “approximately” as applied to different practical situations may have been provided elsewhere in this disclosure.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

For the purposes of this disclosure and the appended claims, the expression of the type “element A and/or element B” is defined to have the meaning that covers embodiments having element A alone, element B alone, or elements A and B taken together and, as such, is intended to be equivalent to “at least one of element A and element B”.

While the invention is described through the above-described specific non-limiting embodiments, it will be understood by those of ordinary skill in the art that modifications to, and variations of, the illustrated embodiments may be made without departing from the inventive concepts disclosed herein. The disclosed aspects may be combined in ways not listed above. Accordingly, the invention should not be viewed as being limited to the disclosed embodiment(s).

What is claimed is:

1. A method comprising:
acquiring a first optical image formed by Raman scattering of excitation light at a first portion of a biological cell culture carried by an aluminum substrate;
acquiring a second optical image formed by said Raman scattering at a second portion of the biological cell culture carried by a passive substrate;
differentiating between a first Raman scattering signal contributed to said Raman scattering by an interior of a cell of the biological cell culture and a second Raman scattering signal contributed to said Raman scattering only by a membrane of the cell based on transforming the first and second optical images with the use of force images of the first portion and the second portion,
wherein the force images have been acquired with the use of a force microscope.

2. A method according to claim 1, wherein the biological cell culture is a culture of cancerous cells.

3. A method according to claim 1, carried out without impairing the membrane and/or without separating the membrane from an interior of the cell.

4. A method according to claim 1, wherein said transforming includes:
subtracting one of the first optical image and the second optical image from the other of the first optical image and the second optical image, and/or
normalizing at least one of the first and second optical images by a height of a peak of a corresponding force image of the force images.

5. A method according to claim 1, comprising:
generating a target image representing only the membrane of the cell of the biological culture.

6. A method according to claim 5, wherein said generating includes generating an image of plasma membrane of an intact cancer cell.

7. A method according to claim 5,
wherein, when the biological cell culture is a cell culture of cancerous cells, said generating includes generating a cancerous target image representing only a membrane of a cancerous cell;
and further comprising:
acquiring a third optical image formed by Raman scattering of excitation light at a third portion of an auxiliary biological cell culture carried by the aluminum substrate, the auxiliary biological culture being a culture of non-cancerous cells,
acquiring a fourth optical image formed by said Raman scattering at a fourth portion of the auxiliary biological cell culture carried by the passive substrate,
differentiating between a third Raman scattering signal contributed to said Raman scattering by an interior of an auxiliary cell of the auxiliary biological cell culture and a fourth Raman scattering signal contributed to said Raman scattering by a membrane of the auxiliary cell based on transforming the third and fourth optical images with the use of force images of the third portion and the fourth portion, wherein the force images have been acquired with the use of the force microscope,
and
generating a non-cancerous target image representing only the membrane of the auxiliary cell of the auxiliary biological cell culture.

8. A method according to claim 7, further comprising:
based on comparison of respectively corresponding lipid-to-protein ratios, derived from the cancerous target image and the non-cancerous target image, generate indicia of protein activity in the cancerous cell.

9. A method according to claim 5,
wherein said target image is an image representing only a membrane of an intact cell of the biological culture, and
further comprising:
assigning a first lipid-to-protein ratio, identified based on the target image in a first portion of the membrane, as a first biomarker,
assigning a second lipid-to-protein ratio, identified based on the target image in a second portion of the membrane that is different from the first portion of the membrane, as a second biomarker, and
based at least on the use of said first and second biomarkers, identifying changes in a biomolecular composition of the membrane that are specific to an identified cancer.

10. A method according to claim 9, further comprising generating indicia of the identified cancer based at least on results of said identifying.

11. A method according to claim 1, further comprising: generating the aluminum substrate by aggregating aluminum nanoparticles with random distribution of dimensions of up to and/or in excess of about 50 nm on a supporting surface to increase enhancement factor of Raman scattering at the first portion of the biological cell culture.

12. A method according to claim 3,
wherein said transforming includes:
subtracting one of the first optical image and the second optical image from the other of the first optical image and the second optical image, and/or
normalizing at least one of the first and second optical images by a height of a peak of a corresponding force image of the force images,
and further comprising:
generating a target image representing only the membrane of the cell of the biological culture, the cell being an intact cell.

13. A method according to claim 12, further comprising:
assigning a first lipid-to-protein ratio, identified based on the target image in a first portion of the membrane, as a first biomarker,
assigning a second lipid-to-protein ratio, identified based on the target image in a second portion of the membrane that is different from the first portion of the membrane, as a second biomarker, and
generating indicia of an identified cancer based on changes of a biomolecular composition of the membrane that are specific to the identified cancer and that are determined from at least the first and second biomarkers.

14. A method according to claim 1, further comprising:
generating a cancerous target image representing only the membrane of the cell of the biological culture, wherein the cell is an intact cancerous cell;
acquiring a third optical image formed by Raman scattering of excitation light at a third portion of an auxiliary biological cell culture carried by the aluminum substrate, the auxiliary biological culture being a culture of non-cancerous cells;
acquiring a fourth optical image formed by said Raman scattering at a fourth portion of the auxiliary biological cell culture carried by the passive substrate;
differentiating between a third Raman scattering signal contributed to said Raman scattering by an interior of an auxiliary cell of the auxiliary biological cell culture and a fourth Raman scattering signal contributed to said Raman scattering by a membrane of an intact auxiliary non-cancerous cell based on transforming the third and fourth optical images with the use of force images of the third portion and the fourth portion, wherein the force images have been acquired with the use of the force microscope;
generating a non-cancerous target image representing only the membrane of the intact auxiliary non-cancerous cell;
and
based on comparison of respectively corresponding lipid-to-protein ratios, derived from the cancerous target image and the non-cancerous target image, generate indicia of changes in a biomolecular composition of the membrane that are specific to an identified cancer.

15. A method according to claim 7,
wherein said target image is an image representing only a membrane of an intact cell of the biological culture, and
further comprising:
assigning a first lipid-to-protein ratio, identified based on the target image in a first portion of the membrane, as a first biomarker,
assigning a second lipid-to-protein ratio, identified based on the target image in a second portion of the membrane that is different from the first portion of the membrane, as a second biomarker, and
based at least on the use of said first and second biomarkers, identifying changes in a biomolecular composition of the membrane that are specific to an identified cancer.
